(19) United States
(12) Patent Application Publication
(10) Pub. No.: US 2003/0100877 A1
(43) Pub. Date: May 29, 2003

(54) Absorbent garment with incorporated skin wellness ingredients

(76) Inventor: Carol L. Erdman, Duluth, GA (US)

Correspondence Address: HUNTON & WILLIAMS, INTELLECTUAL PROPERTY DEPARTMENT, 1900 K STREET, N.W., SUITE 1200, WASHINGTON, DC, 20006-1109, US

(21) Appl. No.: 09/993,884

(22) Filed: Nov. 27, 2001

(51) Int. Cl. A61K 35/80; A61F 13/20; A61K 35/78

(52) U.S. Cl. 604/385.23; 424/195.17; 424/195.18; 424/744; 424/745; 424/769

(57) ABSTRACT

An absorbent garment comprising skin wellness ingredients is disclosed, where the skin wellness ingredients are incorporated into an adhesive used in the assembly of the garment. By incorporating the skin wellness ingredients into the adhesive, the skin wellness ingredients can be precisely placed on those portions of the absorbent garment where at least perceived skin care benefits for the wearer's skin are desired.

### BACKGROUND OF THE INVENTION

1. Field of the Invention

The invention relates generally to the field of absorbent garments and specifically to the field of absorbent garments with incorporated skin wellness ingredients. An absorbent garment with skin wellness ingredients strategically disposed on areas of the garment to provide skin care benefits to the wearer of the garment is disclosed, along with a method and system of manufacture of the absorbent garment. By incorporating skin wellness ingredients into the adhesive used during assembly of the absorbent garment, the skin wellness ingredients can be targeted to those specific areas of the garment to provide, at the least, a perceived skin care benefit while not causing skin irritation or other adverse side effects otherwise experienced by the use of other ingredients added to absorbent garments during their manufacture. The skin wellness ingredients may be placed on the absorbent garments in areas that either contact, or do not contact, the wearer's skin directly, depending on the desired effect of the incorporated skin wellness ingredient.

2. Description of Related Art

Disposable absorbent garments such as infant diapers or training pants, adult incontinence products, and other such products are well-known in the art. Typically, the chassis of such absorbent garments comprises a liquid-permeable body-contacting liner sheet (or “top sheet”), a liquid-impermeable backing sheet (or “back sheet”), and a moisture-absorbent core fiber (or “absorbent core”). The absorbent core usually is made of a nonwoven mat of randomly arrayed fiber and super absorbent polymer (“SAP”) and is generally disposed between the top sheet and the back sheet. In addition, the garment may include waste containment features as leg cuffs or leg gathers, and may also incorporate form fitting elastic elements in the waist, body and leg opening areas.

These garments typically are prepared by continuously supplying the various components of the garment, and forming these components into the final garment. Methods of bonding the different parts of the absorbent garment to form a finished garment are well known in the art. Various attachment mechanisms used for bonding different parts of the absorbent garment to form a finished garment include using a uniform continuous layer of adhesive, a patterned layer of adhesive, or an array of separate lines, spirals, or spots of adhesive. Suitable construction adhesives for attaching the various parts of the garment are manufactured by H.B. Fuller Company of St. Paul, Minn., marketed as HL-1258; Ato Findley Inc. of Wauwatosa, Wis., marketed as Findley 2031 and H2587-01; National Starch Co. of Bridgewater, N.J., marketed as NS34-5665.

An example of a suitable attachment mechanism is one that comprises an open pattern network of filaments of adhesive, as disclosed in U.S. Pat. No. 4,573,986. Another suitable attachment mechanism is one that comprises several lines of adhesive filaments swirled into a spiral pattern, as illustrated by the apparatus and methods shown in U.S. Pat. Nos. 3,911,173, 4,785,996, and 4,842,666. Each of these patents is incorporated herein by reference in its entirety. Alternatively, the attachment mechanism may comprise heat bonds, pressure bonds, chemical or solvent bonding, ultrasonic bonding or welding, stitching, dynamic mechanical bonds, autogenous bonding, or any other suitable attachment mechanism or combinations of these attachment mechanisms as are known in the art.

Methods of bonding elastic elements to garment materials, as for example by spiral spray adhesive application systems, are also well known. Spiral spray adhesive application consists of ejecting a bead of hot melt adhesive, directing jets of pressurized air to form an elongated adhesive fiber from the bead, and imparting a rotational motion to the adhesive fiber. The spirals of adhesive are then deposited on the target substrate, here elastic elements, which then are associated with the garment materials. Such adhesive applications are described, for example, in U.S. Pat. No. 6,235,137, the disclosure of which is herein incorporated by reference in its entirety, and in a manner that is consistent with this disclosure. A suitable adhesive for elastic elements is 34-578A by National Starch Co. of Bridgewater, N.J.

The very nature of the use of absorbent garments causes concern for adverse skin effects to the wearer of the garment. For example, the moisture vapor and heat generated by bodily exudate can accumulate in a pocket of the diaper, becoming trapped and possibly leading to conditions adjacent to a wearer's skin that promote skin irritation, infection, and the like. Although a back sheet, as described above, is generally effective in precluding the passage of bodily exudate outwardly, the hydrophobic nature of the back sheet material can trap the vapor from the bodily exudates, increasing the chance of skin irritation. Further, bodily exudates often produce degradation products that can lead to malodor and general feelings of uncleanliness to the wearer of the garment.

One solution to the problem of adverse skin effects is the addition of different skin emollients, humectants, lotions, bactericides, bacteriostat, antiseptics, perfumes, essential oils, and/or antimicrobial agents to the various nonwoven materials used to form the top sheet, back sheet, and leg cuffs to counteract the malodor problems and protect the skin from the effect of contact with bodily exudate and resulting degradation products. The process of incorporating additives in the nonwoven sheets, however, can be expensive because precise placement of the additives to the nonwoven sheets is not economically feasible for continuous assembly of the garments. This often results in a general coating, or at least a general coating of a certain area, of the nonwoven sheet. Thus, excessive additives may be applied to the components of the garment because they often are incorporated into portions of the garment that contact portions of the wearer's skin where skin care benefits are not needed or desired.

The addition of skin wellness ingredients and fragrances to delivery systems for various surfaces are well known, including the addition of these compositions to adhesive materials. For example, U.S. Pat. No. 5,968,533 issued to Porter et. al., the disclosure of which is herein incorporated by reference in its entirety to the extent that it is consistent with this disclosure, discloses the addition of Vitamins A, C, and E and moisturizers to adhesive materials and applying the adhesive materials to the area of the skin where the treatment is desired. U.S. Pat. No. 4,880,690 issued to Szycher et. al., the disclosure of which is herein incorporated by reference in its entirety to the extent that it is consistent with this disclosure, discloses a perfume patch where a fragrance is added to a polymeric substrate or carrier, which then is attached to an adhesive layer. Migration of the fragrance to the adhesive layer was a noted problem in prior art, which could cause the adhesive to plasticize and leave traces or residue on the surface to which the perfume patch was attached.

The addition of volatile substances to adhesives for delivery to the skin also is known. For example, U.S. Pat. No. 5,603,948 issued to Merkle et. al., the disclosure of which is herein incorporated by reference in its entirety to the extent that it is consistent with this disclosure, discloses a skin patch for the controlled topical or transdermal release of volatile active ingredients, where the active ingredient is dissolved or dispersed homogeneously in an adhesive layer.

In addition, incorporating various ingredients into an absorbent garment is known. For example, certain ingredients have been disclosed as incorporated into various parts of an absorbent garment, principally to provide antimicrobial effects as well as fragrance benefits. Often, these ingredients have been incorporated into the top sheet material. For example, U.S. Pat. Nos. 5,607,760, 5,609,587, 5,635,191, 5,643,588, 5,968,025, and 6,118,041, each of which is issued to Roe et. al., the disclosures of which are herein incorporated by reference in their entirety to the extent that they are consistent with this disclosure, disclose an absorbent garment having a lotioned top sheet, where a plastic or fluid emollient is immobilized on the surface of the top sheet to provide therapeutic or protective lotion coating benefits to the wearer of the garment. Such benefits include reduction of the adherence of bowel movement material to the skin of the wearer. Addition of a hydrophilic surfactant to the top sheet also is disclosed to improve the fluid handling properties of the top sheet.

U.S. Pat. No. 6,149,934 issued to Krzysik et. al., the disclosure of which is herein incorporated by reference in its entirety to the extent that it is consistent with this disclosure, discloses an absorbent garment having a lotion formulation on the outer bodyfacing surface of the garment to act as a lubricant to reduce the abrasion of the skin caused by the liner and also to provide improved skin health to the wearer of the garment. U.S. Pat. No. 6,156,024 issued to Schulte et. al., the disclosure of which is herein incorporated by reference in its entirety to the extent that it is consistent with this disclosure, discloses an absorbent garment having lotioned leg cuffs to minimize the abrasion between the cuffs and the skin where the cuffs contact the wearer's skin, and also to reduce the adherence of bowel movement material to the wearer's skin.

Other documents disclose disposing other types of skin care compositions other than or in addition to emollients and/or lotions to absorbent garments. Such additives typically are intended to enhance skin wellness, to improve the clean up of bowel movement material from the skin of the wearer, to improve breathability of the top sheet of the garment, to provide a skin protective layer to the wearer of the garment, to minimize abrasion to the wearer's skin, or to protect against skin overhydration. See, for example, U.S. Pat. No. 6,204,208 issued to Krzysik et. al.; U.S. Pat. No. 6,166,285 issued to Schulte et. al.; U.S. Pat. No. 6,153,209 issued to Vega et. al.; U.S. Pat. No. 6,120,783 issued to Roe et. al.; U.S. Pat. No. 6,120,488 issued to VanRijswijck et. al., and U.S. Pat. No. 6,107,537 issued to Elder et. al., the disclosures of which are herein incorporated by reference in their entirety to the extent that they are consistent with this disclosure. The disclosed additives include aloe vera, surfactants, zinc oxide, talc, starch, allantoin, hexamidine and its derivatives and salts, tracetin, phytic acid, EDTA (ethylenediamine tetraacetic acid), phenylsulfonylfluorides, chitosan, tocopherol, humectants, anti-oxidants, viscosity modifiers, suspending agents, pH buffering systems, perfumes, soothing agents, pigments, disinfectants, antibacterial additives, pharmaceutical additives, film formers, deodorants, opacifiers, astringents, and solvents. Notably, these additives are disclosed as being added to the nonwoven materials that are used to make the top sheet and back sheet, or rather the leg cuff material, and are promoted as providing a skin barrier benefit or improving the clean up of solid particles from bowel movements from the skin of the wearer of the garment.

Similarly, other documents, for example U.S. Pat. No. 6,215,038 issued to Davis et. al., the disclosure of which is herein incorporated by reference in its entirety to the extent that it is consistent with this disclosure, discloses providing additives to absorbent articles to increase skin hydration by increasing the osmality of a solution outside, but in contact with, the skin. Davis discloses that such additives cause water to move across the skin cellular boundary to dilute the solution, which in turn may reduce redness and irritation to the wearer's skin due to contact with fluids. Other documents, for example U.S. Pat. Nos. 5,938,649 and 5,944,705 each issued to Ducker et. al., the disclosures of which are herein incorporated by reference in their entirety to the extent that they are consistent with this disclosure, disclose the addition of substances, such as aloe vera and a lubricant, to the surface of an absorbent garment to reduce rash to the wearer's skin.

U.S. Pat. No. 6,197,322 issued to Dutkiewicz et. al., the disclosure of which is herein incorporated by reference in its entirety to the extent that it is consistent with this application, discloses the addition of certain antimicrobial agents to the surface of a nonwoven fabric, such as that used in top sheets of absorbent garments, to retard or prevent the growth of bacteria in a personal care article. Other disclosures relate to the addition of odor control materials for preventing or combatting malodorous compounds present in personal care articles in use, such as U.S. Pat. No. 6,080,908 issued to Guarracino et. al., and U.S. Pat. Nos. 5,769,833 and 5,591,146 issued to Hasse, the disclosures of which are herein incorporated by reference in their entirety to the extent that they are consistent with this disclosure.

PCT application WO 99/64083, published Dec. 16, 1999 by Tramontana, and U.S. Pat. No. 6,284,261B1, issued to Tramontana, the disclosures of which are herein incorporated by reference in their entirety to the extent that they are consistent with this disclosure, disclose an absorbent garment containing essential oils added as odor control and antimicrobial agents. Some of the essential oils disclosed in the Tramontana documents include rosemary oil, clove oil, ginger oil, turmeric oil, chamomile oil, lemon grass oil, thyme oil, achillea oil, thulasi oil, clary sage oil, and cedar (hinoki) oil, in an amount of from about 0.1 to about 8 weight % of the absorbent garment.

### SUMMARY OF THE INVENTION

The present inventor has found that many of the absorbent garments assembled according to the documents discussed above have relatively high levels of additives, which makes them expensive to produce. In addition, these levels of additives may cause skin irritation. Thus, there is a need for an absorbent garment having incorporated therein skin wellness ingredients that provide a skin care benefit to the wearer of the garment without causing significant skin irritation. In addition, there is a need for an absorbent garment having incorporated therein skin wellness ingredients that is inexpensive to manufacture.

It is therefore a feature of an embodiment of the invention to provide an absorbent garment with incorporated therein skin wellness ingredients to provide a skin care benefit, or at least a perceived skin care benefit, to the wearer of the garment without causing irritation to the wearer's skin. It is another feature of an embodiment of the invention to provide an absorbent garment having skin wellness ingredients incorporated therein that is relatively inexpensive to manufacture due to the amounts and precise placement of the skin wellness ingredients.

It is yet another feature of an embodiment of the invention to provide an absorbent garment having skin wellness ingredients incorporated therein where the skin wellness ingredients are mixed into the adhesives used during assembly of the garment. It is yet another feature of an embodiment of the invention to provide an absorbent garment having skin wellness ingredients incorporated therein where the skin wellness ingredients can be precisely located on the garment to contact the wearer's skin only in those areas where the skin care benefit, or at least the perceived skin care benefit, of the skin wellness ingredients is desired.

In accordance with these and other features of various embodiments of the invention, there is provided an absorbent article that includes a top sheet material, a back sheet material, and an absorbent core disposed between the top sheet material and the back sheet material. The absorbent article further includes an adhesive, whereby at least some of the adhesive of the absorbent garment contains a skin care benefit effective amount of a skin wellness ingredient.

In accordance with an additional feature of an embodiment of the invention, there is provided a method for making an absorbent garment that includes preparing a top sheet material, preparing a back sheet material, and disposing an absorbent core between the top sheet material and the back sheet material. The method further includes bonding at least the top sheet material to the back sheet material or bonding the absorbent core to at least one of the top sheet material and the back sheet material using at least an adhesive. The method is further characterized in that adhesive used for bonding includes a skin care benefit effective amount of a skin wellness ingredient.

These and other features and advantages of the invention will be apparent to one skilled in the art upon reading the detailed description that follows.

### BRIEF DESCRIPTION OF THE DRAWINGS

The invention will be described with reference to the accompanying drawings, in which like elements are depicted using like numerals.

FIG. 1 is a schematic view of an absorbent garment according to one embodiment of the invention.

FIG. 2 is a side view of the crotch region of the absorbent garment of FIG. 1 along section line A-A.

### DETAILED DESCRIPTION OF THE PREFERRED EMBODIMENTS

“Absorbent garment,” as used herein, refers to articles and garments that absorb and contain body exudates, and more specifically refers to articles and garments that are placed against or in proximity to the body of the wearer to absorb and contain the various exudates discharged from the user's body. A non-exhaustive list of examples of “absorbent garments” includes diapers, diaper covers, disposable diapers, training pants, feminine hygiene products, and adult incontinence products. The invention can be used with all of the foregoing classes of absorbent articles and garments, without limitation, whether disposable or otherwise. Furthermore, the invention will be understood to encompass, without limitation, all classes and types of absorbent articles and garments, including those described above.

Throughout this description, the expressions “skin wellness ingredient” and “skin wellness ingredients” denote those ingredients generally recognized as having some evidence of providing at least a perceived skin care benefit to skin, and those ingredients that in fact have some purported skin care benefits. Throughout this description, the expression “skin care benefit” denotes any benefit to the skin related to the presence of a skin wellness ingredient, and preferably includes a skin care benefit selected from calming, soothing, moisturizing, softening, cooling, coating or other palliative effects on the skin.

Throughout this description, the expression “skin care benefit effective amount” denotes an amount of skin wellness ingredient that is effective in either producing an actual skin care benefit, or is effective in producing a perceived skin care benefit to the wearer of the garment. It is preferred that the skin care benefit effective amount ranges anywhere from about 1% to about 3% by weight of the skin wellness ingredient, based on the weight of the adhesive. When incorporated into an absorbent garment, it is preferred that the skin care benefit effective amount ranges from about 0.0003 weight % to about 0.15 weight % of skin wellness ingredient, preferably from about 0.0005 weight % to about 0.10 weight %, more preferably from about 0.0006 to about 0.09 weight %, even more preferably from about 0.0008 weight % to about 0.05 weight %, and most preferably from about 0.001 weight % to about 0.002 weight %, based on the total weight of the absorbent article. The skin wellness ingredient may be incorporated into all of the adhesive used to assemble the absorbent garment, but preferably is incorporated only into the adhesive used to assemble selected portions of the garment, as for example the transfer layer.

Although the various embodiments of the invention are described in the context of a diaper, it is readily apparent and understood that this is not intended to limit the invention.

The invention relates to an absorbent garment assembled with skin wellness ingredients incorporated into the adhesives used in assembling the garment. Such adhesive application allows for the precise placement of the skin wellness ingredients to those areas of the garment where the skin care benefit, or at least the perceived skin care benefit, for the wearer is desired. Further, such incorporation into the adhesive can be less expensive than incorporating the skin wellness ingredients into the nonwoven materials that make up the top sheet, back sheet, or leg cuffs or gathers. In addition, incorporating the skin wellness ingredients into the adhesive is believed to maintain the level of the skin wellness ingredients low enough that the wearer's skin is not irritated. In addition, the levels of skin wellness ingredients disclosed will not affect the adhesive properties, which could lose tensile strength and stability over time upon the incorporation of high levels of skin wellness ingredients.

It has been found that skin care benefits may also be derived, or may be perceived by the wearer of the garment as derived, from the incorporation of relatively small amounts of skin wellness ingredients into various portions of the absorbent garment, thereby providing calming, soothing, moisturizing, softening, cooling, coating or other palliative effects on the skin. Such skin wellness ingredients may include algae extract, aloe, allantoin, bisabolol, calendula officinalis, Canadian balsam, cocoa butter, colloidal oatmeal, dimethicone, eucalyptus leaf oil, German chamomile, glycerin, Gorgonian extract, helichrysum angustifolium wax, jojoba, lanolin, lavandin, lemon balm, mineral oil, petrolatum, rosemary, spike lavender, tea tree, true lavender, Vitamin E, Vitamin E-acetate, witch hazel, and zinc oxide. These skin wellness ingredients may be incorporated into an adhesive at relatively low levels, and yet may still be effective in causing actual, or at least a perception of, skin care benefits to the garment wearer.

In accordance with one embodiment of the invention, an adhesive having skin wellness ingredients incorporated therein can be used during the assembly of an absorbent garment. Some or all of the parts of the absorbent garment may be joined using an adhesive, including, inter alia, hot melt adhesives such as elastomeric and/or construction adhesives. For example, an adhesive may be used to bond the top sheet and back sheet at their perimeter regions, and this adhesive may include skin wellness ingredients, while the adhesive that may be used to join the absorbent core to the back sheet or to the top sheet may not include any skin wellness ingredients. Alternatively, different parts of the absorbent garment may be operatively associated using different means, as some may be bonded using adhesives with incorporated skin wellness ingredients and some may be joined using chemical or solvent bonding, ultrasonic welding, stitching, heat bonding, or autogenous bonding techniques well known in the art, or by other methods later developed. A plurality of skin wellness ingredients may be incorporated into the adhesive as well, to provide a combination of desired skin care benefits, or at least perceived desired skin care benefits, to the wearer of the absorbent garment.

In one embodiment of the invention, an absorbent garment may be assembled by applying adhesives that include skin wellness ingredients to portions of the garment that will contact the wearer's skin at those points where the skin care benefit is desired. This embodiment of the invention is useful even though the adhesive may not be used to bond one component of the garment to another component. This technique allows the placement of skin wellness ingredients to those parts of the absorbent garment that would not otherwise contain adhesive, without coating the entire component. Accordingly, strategically placing the adhesive enables precise placement of the skin wellness ingredient to the absorbent garment at the desired location, at a much reduced cost, when compared to applying the skin wellness ingredient to the entire sheet of a component of the garment where a majority of the applied skin wellness ingredient would not be beneficial to the skin of the wearer.

While it has previously been known to include certain additives in absorbent garments, these additives typically are present in relatively high weight percentages to achieve their desired effects. Typically, additives were included in amounts from around 0.006 weight % to about 1.5 weight %, based on the weight of the top sheet. The inventor has found that including certain additives at these amounts may cause negative skin effects, such as skin irritation, and the like.

The skin wellness ingredients used in the invention preferably are incorporated into the adhesive before application to the absorbent garment during assembly to achieve the desired skin care benefit to the wearer of the garment. If all of the adhesives used in an absorbent garment are adhesives having skin wellness ingredients incorporated therein, and the skin wellness ingredients are present in an amount of about 3 weight % based upon the weight of the adhesive, then the overall absorbent garment may include up to about 0.15 weight % of skin wellness ingredient(s) in the final garment, based on the total weight of the final garment. More typically, skin wellness ingredient-incorporated adhesive would only be used in certain portions of the garment components, reducing the total amount of skin wellness ingredient in the final garment to well less than about 0.15 weight %, based on the total weight of the final garment.

Those skilled in the art will recognize that using only certain adhesives having skin wellness ingredients incorporated therein in the construction of the absorbent garment will result in a lower amount of skin wellness ingredients in the final absorbent garment. For example, if the construction adhesive was the only skin wellness ingredient-incorporated adhesive used, then the overall amount of skin wellness ingredient in the absorbent garment may be from about 0.03 weight % to about 0.10 weight %, based on the total weight and design of the garment, assuming all of the construction adhesive contained from 1 to 3 weight % skin wellness ingredient. Comparatively, using skin wellness ingredient-incorporated adhesive only in the adhesive used to bond the transfer layer may result in a final level of skin wellness ingredient in the absorbent garment from about 0.0003 weight % up to about 0.001 weight % based on the total weight of the garment, again assuming the adhesive contained from 1 to 3 weight % skin wellness ingredient. Similarly, using skin wellness ingredient-incorporated adhesive only in the elastics adhesive may result in a final level of skin wellness ingredient in the absorbent garment from about 0.02 weight % up to about 0.06 weight %, based on the total weight of the garment, assuming the adhesive contained from 1 to 3 weight % skin wellness ingredient.

The amounts of adhesive may vary, depending on which components of the garment use skin wellness ingredient-incorporated adhesive, what areas of each component the skin wellness ingredient-incorporated adhesive is applied to, and how much of the skin wellness ingredient is incorporated into the adhesive. For example, only the elastics adhesive used to bond the elastics in the leg cuff area may contain skin wellness ingredients, whereas the elastics adhesive used in the waist area may not. These relatively small amounts of skin wellness ingredients result in significant cost savings in the manufacture of the absorbent garment, while still providing a skin care benefit to the wearer of the garment, either actual or perceived.

The skin wellness ingredients may be added to virtually any hot melt type adhesive. Any base polymer suitable for use in formulating hot melt adhesives, as are well known to those skilled in the art, may be used in the practice of the invention. Such polymers include amorphous polyolefins, ethylene-containing polymers and rubbery block copolymers, as well as blends thereof. Hot melt adhesive compositions based on ethylene/vinyl acetate copolymers, isotactic or atactic polypropylene, styrene-butadiene, styrene-isoprene, or styrene-ethylene-butylene A-B-A or A-B-A-B block copolymers or mixtures thereof may be used. In addition to the base polymer, the hot melt adhesive compositions may also contain tackifiers, oils and/or waxes as well as conventional additives including stabilizers, anti-oxidants, pigments and the like.

For example, hot melt adhesives may be styrene-butadiene block copolymers, as disclosed in U.S. Pat. No. 4,944,993 issued to Raykovitz et. al. Hot melt adhesives including additives are disclosed in U.S. Pat. No. 4,526,577 issued to Schmidt, Jr. et. al. and U.S. Pat. No. 5,603,948 issued to Merkle et. al. The disclosures of these documents are incorporated herein by reference in their entirety to the extent that they are consistent with this disclosure.

Acceptable construction adhesives in which skin wellness ingredients may be incorporated for use in the invention include HL-1258 by H.B. Fuller Company of St. Paul, Minn.; Findley 2031 and H2587-01 by Ato Findley Inc. of Wauwatosa, Wis.; and NS34-5665 by National Starch Co. of Bridgewater, N.J. Acceptable adhesives that may include skin wellness ingredients to be used to secure elastic elements to the absorbent garment for use in the invention include 34-578A by National Starch Co. of Bridgewater, N.J.

It has been found that certain skin wellness ingredients may be slightly more effective when incorporated into certain types of adhesives. For example, lavender oil may work better when added to a construction adhesive than when added to an elastomeric adhesive, although incorporating lavender oil into elastomeric adhesive will still provide acceptable performance for purposes of the invention. One skilled in the art can determine the most effective adhesive to use for different skin wellness ingredients without undue experimentation.

Some of the skin wellness ingredients may require modification from their substantially pure state before being added to the adhesive for use according to the invention so that these skin wellness ingredients can retain their respective skin care benefits throughout the adhesive application process. Those skilled in the art can determine without undue experimentation those skin wellness ingredients that may require such modification, and also the appropriate modification for the skin wellness ingredients to remain effective according to the invention through the conditions incurred in adhesive application to the various components of the absorbent garment.

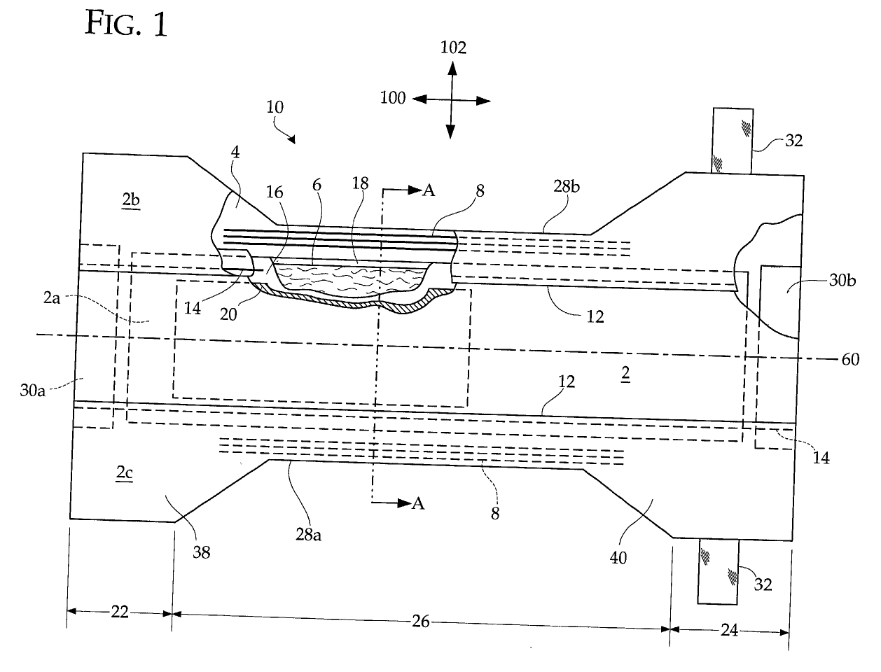

Turning now to the drawings, a preferred embodiment of the invention comprises a disposable absorbent garment 10 of the diaper type, such as shown, for example, in FIG. 1. It should be understood, however, that the invention is applicable to other types of absorbent garments. With reference to FIG. 1, the diaper 10 according to a first preferred embodiment is shown in a relaxed condition with the effects of the elastics removed for purposes of clarity in the description. The diaper 10 has a generally hourglass shape and can generally be defined in terms of a front waist region 22, a back waist region 24, and a crotch region 26. Those skilled in the art will recognize that “front” and “back” are relative terms, and these regions may be transposed without leaving the scope of the invention. Alternatively, the diaper can be configured in a generally rectangular shape or in a “T” shape. A pair of leg openings 28a, 28b extend along at least a portion of the crotch region 26. The diaper preferably comprises a top sheet 2, a back sheet 4, which may be substantially co-terminus with the top sheet 2, and an absorbent core laminate 6 disposed between at least a portion of the top sheet 2 and back sheet 4. One or more pairs of leg elastics 8 (three pairs are shown in FIG. 1) extend adjacent to leg openings 28a, 28b, respectively.

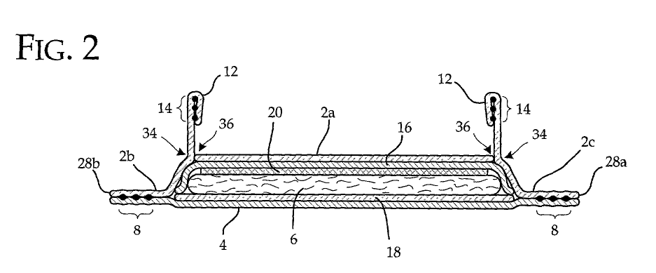

The diaper may further include a front waist elastic system 30a, a back waist elastic system 30b, a fastening system 32 (e.g., tape or other suitable mechanical fastener) and a waste containment system in the form of waste containment flaps 12 (also known as standing leg gathers). Waste containment flaps 12 (FIG. 2) preferably extend from the front waist region 22 to the back waist region 24 along opposite sides of a longitudinal center line or axial center line 60 of the diaper 10, or alternatively only along a portion thereof. The front waist region 22 and rear waist region 24 may include ear portions 38, 40 extending outwardly from the leg openings 28a, 28b.

A variety of back sheet and top sheet constructions and materials are available and known in the art, and the invention is not intended to be limited to any specific materials or constructions of these components. The back sheet 4 is of any suitable pliable liquid-impervious material known in the art. Typical back sheet materials include films of polyethylene, polypropylene, polyester, nylon, and polyvinyl chloride and blends of these materials. For example, the back sheet can be of a pigmented polyethylene film having a thickness in the range of 0.02-0.04 mm. The moisture-pervious top sheet 2 can be of any suitable relatively liquid-pervious material known in the art that permits passage of liquid therethrough. Non-woven top sheet materials are exemplary because such materials readily allow the passage of liquids to the underlying absorbent core 6. Examples of suitable top sheet materials include non-woven spunbond or carded webs of polypropylene, polyethylene, nylon, polyester and blends of these materials.

The back sheet 4 and the top sheet 2 are “associated” with one another. The term “associated” encompasses configurations whereby the top sheet 2 is directly joined to the back sheet 4 by affixing the top sheet 2 directly to the back sheet 4, and configurations whereby the top sheet 2 is indirectly joined to the back sheet 4 by affixing the top sheet 2 through intermediate members which in turn are affixed to the back sheet 4. While the back sheet 4 and top sheet 2 in the preferred embodiment have substantially the same dimensions, they may also have different dimensions.

The top sheet 2 and the back sheet 4 may be associated with one another by a variety of methods known in the art, including, but not limited to: using adhesives such as hot melt adhesives; chemical or solvent bonding; ultrasonic welding; stitching; heat bonding; autogenous bonding; or any other method of affixation known or hereafter discovered. U.S. Pat. No. 4,919,738 issued to Ball et. al. discloses a method of autogenous bonding, and its disclosure is herein incorporated by reference in its entirety in a manner that is consistent with the invention. The adhesive may or may not include skin wellness ingredients, according to the desired properties of the particular absorbent garment being assembled. The skin wellness ingredient may be incorporated into the adhesive at levels ranging from 1 to 3 weight % of the adhesive, depending on the desired properties of the absorbent garment and the nature of the incorporated skin wellness ingredient.

In addition, the back sheet 4 may be covered with a fibrous, nonwoven fabric such as is disclosed for example in U.S. Pat. No. 4,646,362, which is hereby incorporated by reference and in a manner consistent with this disclosure. Materials for such a fibrous outer liner include a spun-bonded nonwoven web of synthetic fibers such as polypropylene, polyethylene or polyester fibers; a nonwoven web of cellulosic fibers, textile fibers such as rayon fibers, cotton and the like, or a blend of cellulosic and textile fibers; a spun-bonded nonwoven web of synthetic fibers such as polypropylene; polyethylene or polyester fibers mixed with cellulosic, pulp fibers, or textile fibers; or melt blown thermoplastic fibers, such as macro fibers or micro fibers of polypropylene, polyethylene, polyester or other thermoplastic materials or mixtures of such thermoplastic macro fibers or micro fibers with cellulosic, pulp or textile fibers. Alternatively, the back sheet 4 may comprise multiple panels, such as three panels wherein a central poly back sheet panel is positioned adjacent the absorbent core 6 while outboard non-woven breathable side back sheet panels are attached to the side edges of the central poly back sheet panel. The back sheet 4 may also be formed from microporous poly coverstock for added breathability. In other embodiments, the back sheet 4 may be a laminate of several sheets. The back sheet 4 may further be treated to render it hydrophilic or hydrophobic, and may have one or more visual indicators associated with it, such as labels indicating the front or back of the diaper or other characters or colorations.

The top sheet 2 may be formed from one or more panels of material and may comprise a laminated sheet construction. In the embodiment of FIG. 1, the top sheet 2 comprises three separate portions or panels. A three-panel top sheet 2 may comprise a central top sheet panel formed from preferably a liquid-pervious material that is either hydrophobic or hydrophilic. The central top sheet panel 2a may be made from any number of materials, including synthetic fibers (e.g., polypropylene or polyester fibers), natural fibers (e.g., wood or cellulose), apertured plastic films, reticulated foams and porous foams to name a few. One preferred material for a central top sheet panel 2a is a cover stock of single ply non-woven material which may be made of carded fibers, either adhesively or thermally bonded, perforated plastic film, spunbonded fibers, or water entangled fibers, which generally weigh from 0.3-0.7 oz./sq. yd. and have appropriate and effective machine direction and cross-machine direction strength suitable for use as a baby diaper cover stock material, as are known in the art. The central top sheet 2a panel preferably extends from substantially the front waist region 22 to the back waist region 24 or a portion thereof.

The second and third top sheet panels 2b, 2c in this embodiment may be positioned laterally outside of the central top sheet panel 2a. The outer top sheet panels 2b, 2c are preferably substantially liquid-impervious and hydrophobic, preferably at least in the crotch area. The outer edges of the outer top sheet panels 2b, 2c may substantially follow the corresponding outer perimeter of the back sheet 4. The material for the outer top sheet panels 2b, 2c is preferably polypropylene and can be woven, nonwoven, spunbonded, carded or the like, depending on the application.

An inner region 34 (FIG. 2) of the outer top sheet panels 2b, 2c preferably are attached by, e.g., an adhesive, to the outer edges 36 of the inner top sheet panel 2a. The adhesive may or may not include skin wellness ingredients, according to the desired properties of the particular absorbent garment being assembled. The skin wellness ingredients may be incorporated into the adhesive at levels ranging from 1 to 3 weight % of the adhesive, depending on the desired properties of the absorbent garment and the nature of the incorporated skin wellness ingredients. At the point of connection with the outer edges 36 of the inner top sheet panel 2a, the inner regions 34 of the outer top sheet panels 2b, 2c extend upwardly to form waste containment flaps 12. The waste containment flaps 12 may be formed of the same material as the outer top sheet panels 2b, 2c, as in the embodiment shown. The waste containment flaps 12 may also be formed from separate elasticized strips of material that are associated with the top sheet, back sheet or both, or otherwise integrated into the garment.

The waste containment flaps 12 may be treated with a suitable surfactant to modify their hydrophobicity/hydrophilicity or imbue them with skin wellness products as desired. The central top sheet panel 2a may extend past the connection point with the waste containment flaps 12 and even extend to the periphery of the back sheet. Still further, the central top sheet panel 2a could extend fully between the outer top sheet panels 2b, 2c and even beyond so that the outer edges 36 of the central top sheet panel 2a are coextensive with and sandwiched between the outer top sheet panels 2b, 2c and the back sheet 4.

Each waste containment flap 12 preferably includes a portion that folds over onto itself to form an enclosure. One or more elastic members 14 (FIG. 2) may be secured in the enclosure in a stretched condition with adhesive, which may or may not include skin wellness ingredients, according to the desired properties of the particular absorbent garment being assembled. The skin wellness ingredients may be incorporated into the adhesive at levels ranging from 1 to 3 weight % of the adhesive, depending on the desired properties of the absorbent garment and the nature of the incorporated skin wellness ingredients. As has been known at least as long the disclosure of Tetsujiro, Japanese Patent document 40-11543, when the flap elastic 14 attempts to assume the relaxed, unstretched condition, the waste containment flaps 12 rise above the surface of the central top sheet portion or panel 2a.

The waist elastics 30a, 30b may be similar structures or different to impart similar or different elastic characteristics to the front and back waist portions 22, 24 of the diaper 10. In general, the waist elastics may comprise elastically extensible foam strips positioned at the front and back waist sections 22, 24. The foam strips are preferably about 0.50 inches to about 2.50 inches wide and about 3 inches to about 6 inches long. The foam strips are preferably positioned between the top sheet panels 2a, 2b, 2c and the back sheet 4. Alternatively, a plurality of elastic strands may be employed as waist elastics rather than foam strips. The foam strips are preferably polyurethane, but could be any other suitable material that preferably decreases waist band roll over, reduces leakage over the waist ends of the absorbent garment, and generally improves comfort and fit. The front and back waist foam strips 30a, 30b are stretched 50-150%, preferably 100% before being adhesively secured between the back sheet 4 and top sheet 2, which adhesive may or may not include skin wellness ingredients, according to the desired properties of the particular absorbent garment being assembled. The skin wellness ingredients may be incorporated into the adhesive at levels ranging from 1 to 3 weight % of the adhesive, depending on the desired properties of the absorbent garment and the nature of the incorporated skin wellness ingredients.

In any or all of the foregoing embodiments, the top sheet 2 may comprise a single sheet of material having different characteristics (e.g., liquid-imperviousness/perviousness and/or hydrophobicity/hydrophilicity) and have regions of transition or demarcation therebetween.

Each leg opening 28a, 28b may be provided with a leg elastic containment system 8, sometimes referred to as conventional leg gathers. In a preferred embodiment, three strands of elastic threads are positioned to extend adjacent the leg openings 28a, 28b between the outer top sheet panels 2b, 2c and the back sheet 4. The selection of appropriate elastics and the construction of leg elastic containment systems is known in the art. For example, the leg elastics 8 may be ultrasonically bonded, heat/pressure sealed using a variety of bonding patterns, or glued to the diaper 10 with adhesive, which adhesive may or may not include skin wellness ingredients, according to the desired properties of the particular absorbent garment being assembled. The skin wellness ingredients may be incorporated into the adhesive at levels ranging from 1 to 3 weight % of the adhesive, depending on the desired properties of the absorbent garment and the nature of the incorporated skin wellness ingredients.

Various commercially available materials may be used for the leg elastics 8 and elastic members 14, such as natural rubber, butyl rubber or other synthetic rubber, urethane, elastomeric materials such as spandex, which is marketed under various names, including LYCRA™ (DuPont), GLOSPAN™ (Globe) and SYSTEM 7000™ (Fulflex), and so on. The invention is not limited to any particular elastic.

The fastening system of the diaper 10 may be attached to the back waist region 24, and preferably comprises tape tabs or mechanical fasteners 32. However, any fastening known in the art will be acceptable. Moreover, the fastening system may include a reinforcement patch below the front waist portion so that the diaper may be checked for soiling without compromising the ability to reuse the fastener. Alternatively, other diaper fastening systems are also possible, including safety pins, buttons, and snaps. Fastening systems are known in the art, and the invention is not limited to using any particular fastening, and may be constructed without any fastening system at all, such as in training pant-type garments.

As stated previously, the invention has been described in connection with a diaper. The invention, however, is not intended to be limited to application only in diapers. Specifically, the invention may be readily adapted for use in other absorbent garments besides diapers, including, but not limited to, training pants, feminine hygiene products and adult incontinence products.

The underlying structure beneath the top sheet 2 may include, depending on the diaper construction, various combinations of elements, but in each embodiment, it is contemplated that the absorbent garment will preferably include an absorbent core 6. The absorbent core 6 may be comprised of one or more layers of material, such as an absorbent layer for storing fluids and an acquisition layer for distributing fluids. Such multiple layer absorbent cores are known in the art and disclosed in U.S. Pat. No. 5,439,458 issued to Noel et al., which is incorporated herein by reference in its entirety, and in a manner consistent with this disclosure.

The absorbent core 6 may be made from any absorbent material or materials known in the art. In one embodiment of the invention, the absorbent core 6 comprises wood fibers or other fibers such as chemical wood pulp, fibrous absorbent gelling material, or any other suitable liquid absorbing material, such as commercially available fluff pulp or fluffed bleached kraft softwood pulp or fibrous absorbent gelling material. In another embodiment of the invention, the absorbent core 6 comprises a combination of a porous fibrous web and super absorbent particles. Absorbent cores are known in the art and are disclosed, for example, in U.S. Pat. No. 5,281,207 issued to Chmielewski et al., U.S. Pat. No. 4,610,678 issued to Weisman et. al., U.S. Pat. No. 5,137,537 issued to Herron et. al., and U.S. Pat. No. 5,147,345 issued to Young et. al., which are incorporated herein by reference in their entirety in a manner consistent with this disclosure. In such an embodiment, the absorbent core 6 may be surrounded by a liquid pervious tissue over-wrap 16, 18, or other material.

The absorbent core 6generally is elongated along the longitudinal axis 100 of the garment, and may extend along either or both of the lateral and longitudinal axes 102, 100 to the outer perimeter of the garment. In the embodiment depicted in FIG. 1, the absorbent core 6 is substantially rectangular in shape, however, it may also have rounded ends or other shapes, such as an “I” shape or a “T” shape. The absorbent core 6 may also have channels, grooves or pockets, and may have a varying thickness. The shape of the absorbent core 6 may be selected to provide the greatest absorbency in target areas where body fluids are most likely to strike the diaper 10, which is often referred to as zoned absorbency.

The absorbent core 6 may be associated with the top sheet 2, the back sheet 4, or any other suitable part of the garment 10 by any method known in the art, in order to fix the absorbent core 6 in place. Preferably, the absorbent core 6 is glued to the diaper 10 with adhesive, which adhesive may or may not include skin wellness ingredients, according to the desired properties of the particular absorbent garment being assembled. The skin wellness ingredients may be incorporated into the adhesive at levels ranging from 1 to 3 weight % of the adhesive, depending on the desired properties of the absorbent garment and the nature of the incorporated skin wellness ingredients.

As shown in FIGS. 1 and 2, an additional layer 20 may be disposed between the top sheet 2 and absorbent core 6, and/or other additional layers may be disposed between these layers, or between absorbent core 6 and back sheet 4. The additional layer can be any layer selected from a fluid acquisition layer, a distribution layer, an additional fibrous layer optionally containing SAP, a wicking layer, a storage layer, or combinations and fragments of these layers. Such layers may be provided to assist with transferring fluids to the absorbent core 6, handling fluid surges, preventing rewet, containing absorbent material, improving core stability, or for other purposes. Skilled artisans are familiar with the various additional layers that may be included in absorbent article, and the invention is not intended to be limited to any particular type of materials used for those layers. Rather, the invention encompasses all types of wicking layers, all types of distribution layers, etc., to the extent that type of layer 20 is utilized.

The additional layers 20 may be bonded to the top sheet 2, the absorbent core 6, other additional layers 20, or any other suitable part of the garment 10 by any method known in the art, in order to fix the additional layers 20 in place. Preferably, the additional layers 20 are glued to the diaper 10 with adhesive, which adhesive may or may not include skin wellness ingredients, according to the desired properties of the particular absorbent garment being assembled. The skin wellness ingredients may be incorporated into the adhesive at levels ranging from 1 to 3 weight % of the adhesive, depending on the desired properties of the absorbent garment and the nature of the incorporated skin wellness ingredients.

One element that is useful as an additional layer 20 in the absorbent article 10 of the invention is a fluid acquisition layer, or fluid handling layer. The fluid acquisition layer 20 typically comprises a hydrophilic fibrous material, and serves to quickly collect and temporarily hold discharged body fluid. A portion of discharged fluid may, depending upon the wearer's position, permeate the acquisition layer 20 and be absorbed by the absorbent core 6 in the area proximate to the discharge. However, since fluid is frequently discharged in gushes, the absorbent core 6 in such area may not absorb the fluid as quickly as it is discharged. Therefore, the fluid acquisition layer 20 hereof also facilitates transport of the fluid from the point of initial fluid contact to other parts of the absorbent core 6. In the context of the invention, it should be noted that the term “fluid” includes, but is not limited to, liquids, urine, menses, perspiration, and water-based body fluids.

The function of the fluid acquisition layer 20 is relatively important. The fluid acquisition layer 20 preferably has sufficient capillary suction to more fully drain the top sheet 2 and yet not exhibit excessive fluid retention to make it difficult for the fibrous structure to desorb the acquisition layer 20. The acquisition layer 20 may be comprised of several different materials including nonwoven or woven webs of synthetic fibers including polyester, polypropylene, or polyethylene, natural fibers including cotton or cellulose, blends of such fibers, foams, fluff pulp, apertured films, or any equivalent materials or combinations of materials.

Another useful layer 20 for use in the absorbent garment 10 of the invention includes a fluid distribution layer 20. Fluid distribution layer 20 of the invention can include any combination or all of three basic components: chemically stiffened, twisted, and curled bulking fibers, high surface area fibers, and binder fibers. In a preferred embodiment of the invention, fluid distribution layer 20 comprises from about 20% to about 80% of the chemically stiffened, twisted, and cured fibers, from about 10% to about 80% of a high surface area fiber, and from 0% to about 50% of a thermoplastic binding means for increasing physical integrity of the web. All percentages herein refer to weight percentages based on total dry web weight. Preferably, the fluid distribution layer 20 will comprise between about 45% and about 60% of chemically stiffened, twisted, and cured fibers, between about 5% and about 15% of a hot melt fibrous binding means, and between about 30% and about 45% high surface area cellulose binding means. More preferably, the fluid distribution layer 20 comprises about 10% thermoplastic binding means, about 45% chemically stiffened, twisted, and cured fibers, and about 45% high surface area fibers.

Chemical additives also can be used as binding means, and are incorporated into the acquisition/distribution layer at levels typically of about 0.2% to about 2.0%, dry web weight basis. The three basic fiber components are described in greater detail in U.S. Pat. No. 5,549,589, the disclosure of which is incorporated by reference herein in its entirety, and in a manner consistent with this disclosure.

Fluid distribution layer 20 also may be comprised of non-woven or woven webs of synthetic fibers, natural fibers, foams, carded, thermal bonded materials, and the like.

Another useful layer in the absorbent article 10 of the invention includes a storage layer 20. Such storage layers 20 typically have limited transport and wicking capabilities but high storage or retention capacity, and rely upon the fibrous structure of the absorbent core 6 to distribute incoming fluid over a larger area. It is preferred to dispose storage layer 20 between the absorbent core 6 and the back sheet 4, or between the first and second tissue layers 16, 18.

Storage layers or members 20 may be of generally conventional design and composition, selected with regard to the particular application. The storage layer or member 20 may be monolayer or multilayer, homogeneous or stratified, profiled or uniform, etc. Materials suitable for use in such storage layers 20 may be natural or synthetic in origin, woven, non-woven, fibrous, cellular, or particulate, and may include particles, layers, or regions of absorbent polymeric gelling materials. Other preferred materials include fluff pulp and SAP composites, either air laid or wet laid, and high capacity resilient foam materials. Storage layer 20 may also have any desired size and/or shape as may prove suitable for a particular application, including square, rectangular, oval, elliptical, oblong, etc. They may also take on a three-dimensional shape or may be substantially planar in nature.

Another useful layer 20 in absorbent article 10 is a wicking layer 20. Wicking layers usually have both fluid acquisition and fluid distribution properties. For example, vertical wicking, which is in general the ability to transport fluids vertically from the top sheet 2 to the absorbent core 6, is related in many respects to fluid acquisition. Horizontal wicking, which is in general the ability to transport fluids along the longitudinal 100 and lateral 102 axes of FIG. 1, is related in many respects to fluid distribution.

Any conventional wicking materials can be used for the wicking layer 20 of the invention. Typical suitable wicking layer 20 materials include, for example, high-density air laid fluff pulps, high-density wet laid fluff pulp, multi-groove fibers, and the like. In addition, high internal phase emulsion (HIPE) foams such as those disclosed in U.S. Pat. No. 5,650,222 can be used, braided materials such as those disclosed in H1,585, and other conventional fibrous and strand materials can be used. The disclosures of U.S. Pat. No. 5,650,222 and H1,585 are incorporated by reference here in their entirety, and in a manner consistent with the invention. Wicking layer 20 also may be comprised of two or more sublayers containing absorbent materials with differing wicking characteristics. Those skilled in the art will be able to include a suitable wicking layer 20 in an absorbent garment manufactured according to the invention without undue experimentation based on the teachings herein.

Various combinations of any of the above-mentioned layers also may be used as the additional layer 20. For example, additional layer 20 may comprise a combination of a wicking layer and a distribution layer, thereby imparting the additional layer 20 with both wicking and distribution properties. Skilled artisans will be capable of designing additional layers 20 to have desired properties by combining various layer attributes, or by fragmenting the layer.

The dimensions of additional layer(s) 20 may be the same as or different from the dimensions of the absorbent core 6 and/or top sheet 2 and the back sheet 4. It is preferred that additional layer(s) 20 have a width in the lateral direction (102) of anywhere from about 10 mm to about 100 mm, and preferably from about 25 mm to about 80 mm.

An exemplary list of skin wellness ingredients that may be incorporated into various hot melt adhesives such as elastomeric and/or construction adhesives for use in bonding parts of absorbent garments to provide a skin care benefit, or at least a perceived skin care benefit, to the wearer of the garment are detailed below, along with that skin care benefit. Those skilled in the art are capable of incorporating any and all of the skin wellness ingredients described in Table 1 below in an adhesive for use in an absorbent garment. Skilled artisans also are capable of including the adhesive having the skin wellness ingredient incorporated therein in any component part of an absorbent garment to achieve the desired skin care benefit.

TABLE 1

| SKIN | | | | ANTI- | |
|---|---|---|---|---|---|
| WELLNESS | COMMON | | ESSENTIAL | MICROBIAL | OTHER |
| INGREDIENT | NAME | FRAGRANCE | OIL | PROPERTIES | PROPERTIES |
| Algae Extract | | | | Possible | 1; 2; 3; 5 |
| Aloe | Aloe | | | ✓ | 2; 4 |
| Barbadensis | | | | | |
| Allantoin | | | | | 1 |
| Bisabolol | | | | | 4 |
| Calendula | Marigold | ✓ | ✓ | | 4 |
| Officinalis | | | | | |
| Flower Oil | | | | | |
| Abies Balsamea | Canadian | ✓ | | ✓ | |
| | Balsam | | | | |
| Theobroma | Cocoa | | | | 1 |
| Cacao Seed | Butter | | | | |
| Butter | | | | | |
| Colloidal | | | | | 1 |
| Oatmeal | | | | | |
| Dimethicone | | | | | 1 |
| Eucalyptus | Eucalyptus | ✓ | ✓ | ✓ | |
| Globulus | Leaf Oil | | | | |
| Matricaria | Chamomile | ✓ | ✓ | Bactericidal | |
| Recutica | | | | | |
| Glycerin | | | | | 1; 3 |
| Gorgonian | Sea Whip | | | | 5 |
| Extract | Extract | | | | |
| (Pseudotergoria | | | | | |
| Elisabethae) | | | | | |
| Helichrysum | | | | | 1 |
| Angustifolium | | | | | |
| Wax | | | | | |
| Jojoba | | | | | 6 |
| Lanolin | | | | | 1 |
| Lavandula X | Lavandin | ✓ | | Possible | 1 |
| Intermedia | | | | | |
| Melissa | Lemon | ✓ | ✓ | | |
| Officinalis | Balm | | | | |
| Mineral Oil | | | | | 1 |
| Petrolatum | | | | | 1 |
| Rosmarinus | Rosemary | ✓ | ✓ | ✓ | |
| Officinalis | | | | | |
| Lavandula | Spike | ✓ | | Possible | |
| Latifolia | Lavender | | | | |
| Melaleuca | Tea Tree | ✓ | | ✓ | |
| Alternifolia | | | | | |
| Lavandula | True | ✓ | ✓ | ✓ | 1 |
| Angustifolia | Lavender | | | | |
| Flower Wax | | | | | |
| Vitamin E | | | | | 6 |
| Vitamin E- | | | | | 4 |
| acetate | | | | | |
| Hamamelis | Witch | Possible | | | 1 |
| Virginiana | Hazel | | | | |
| Zinc Oxide | | | | | 1 |

¹The following properties were taken from the International Cosmetic Dictionary and Handbook, 8^{th} ed. (2000), published by the Cosmetics, Toiletries and Fragrance Association.
1 Skin Protectant
2 Skin Conditioning Agent (Emollient)
3 Skin Conditioning Agent (Humectant)
4 Skin Conditioning Agent (Miscellaneous)
The following property was taken from general anecdotal literature, supporting at least some evidence that an additive has shown some healing activity.
5 Healing Properties
The following property is generally accepted in the art as having some skin care benefit.
6 Skin Care Benefit
It is to be understood that the embodiments and variations shown and described herein are merely illustrative of the principles of this invention and that various modifications may be implemented by those skilled in the art without departing from the scope and spirit of the invention.

The following property was taken from general anecdotal literature, supporting at least some evidence that an additive has shown some healing activity.

5. Healing Properties

The following property is generally accepted in the art as having some skin care benefit.

6. Skin Care Benefit

It is to be understood that the embodiments and variations shown and described herein are merely illustrative of the principles of this invention and that various modifications may be implemented by those skilled in the art without departing from the scope and spirit of the invention.

What is claimed is:

1. An absorbent article, comprising:
a top sheet material;
a back sheet material;
an absorbent core disposed between the top sheet material and the back sheet material; and
adhesive,
wherein at least some of the adhesive comprises a skin care benefit effective amount of a skin wellness ingredient.

2. The absorbent article of claim 1, wherein the skin wellness ingredient is selected from the group consisting of algae extract, aloe, allantoin, bisabolol, calendula officinalis, Canadian balsam, cocoa butter, colloidal oatmeal, dimethicone, eucalyptus leaf oil, German chamomile, glycerin, Gorgonian extract, helichrysum angustifolium wax, jojoba, lanolin, lavandin, lemon balm, mineral oil, petrolatum, rosemary, spike lavender, tea tree, true lavender, Vitamin E, Vitamin E-acetate, witch hazel, or zinc oxide, and mixtures thereof.

3. The absorbent article of claim 1, wherein the amount of the skin wellness ingredient in the absorbent article is from about 1 weight % to about 3 wt %, based on the weight of the adhesive into which the skin wellness ingredient is incorporated.

4. The absorbent article of claim 1, wherein the amount of the skin wellness ingredient present in the absorbent article ranges from about 0.0003 weight % to about 0.15 weight %, based on the weight of the absorbent article.

5. The absorbent article of claim 1, wherein the adhesive bonds the top sheet material to the back sheet material, and the adhesive comprises a skin care benefit effective amount of a skin wellness ingredient.

6. The absorbent article of claim 1, wherein the adhesive bonds the absorbent core to either the top sheet material or the back sheet material, and the adhesive comprises a skin care benefit effective amount of a skin wellness ingredient.

7. The absorbent article of claim 1, further comprising at least one elastic element disposed between the top sheet material and the back sheet material.

8. The absorbent article of claim 7, wherein the adhesive bonds the at least one elastic element to at least one of the top sheet material, the back sheet material, and the absorbent core, and the adhesive comprises a skin care benefit effective amount of a skin wellness ingredient.

9. The absorbent article of claim 1, wherein at least some of the adhesive comprises at least one material selected from the group consisting of styrene-butadiene block copolymer, tackifying resin, oil diluent, and substantially radial styrene-butadiene block copolymer, or triple-block copolymer of polystyrene block copoly(ethylene/butylene) block polystyrene, and mixtures thereof.

10. An absorbent article, comprising:
a top sheet material;
a back sheet material operatively associated with the top sheet material;
an absorbent core operatively associated with at least one of the top sheet material and the back sheet material; and
at least one elastic element operatively associated with at least one of the top sheet material, the back sheet material, and the absorbent core,
wherein the absorbent article further comprises an adhesive with a skin care benefit effective amount of a skin wellness ingredient to produce a skin care benefit for the wearer of the absorbent article.

11. The absorbent article of claim 10, wherein the adhesive is used to operatively association one or more components selected from the group consisting of: the back sheet material to the top sheet material; the absorbent core to at least one of the top sheet material and the back sheet material; and the elastic elements to at least one of the top sheet material, the back sheet material, and the absorbent core.

12. The absorbent article of claim 10, wherein the skin wellness ingredient is selected from the group consisting of algae extract, aloe, allantoin, bisabolol, calendula officinalis, Canadian balsam, cocoa butter, colloidal oatmeal, dimethicone, eucalyptus leaf oil, German chamomile, glycerin, Gorgonian extract, helichrysum angustifolium wax, jojoba, lanolin, lavandin, lemon balm, mineral oil, petrolatum, rosemary, spike lavender, tea tree, true lavender, Vitamin E, Vitamin E-acetate, witch hazel, or zinc oxide, and mixtures thereof.

13. The absorbent article of claim 10, wherein the skin wellness ingredient is present in an amount of from about 0.0003 weight % to about 0.15 weight %, based on the weight of the absorbent article.

14. A method for making an absorbent garment, comprising:
preparing a top sheet material;
preparing a back sheet material;
preparing an absorbent core;
disposing the absorbent core between the top sheet material and the back sheet material;
adhesively bonding the top sheet material to the back sheet material; and optionally
adhesively bonding the absorbent core to at least one of the top sheet material and the back sheet material,
wherein the adhesive used for adhesive bonding comprises a skin care benefit effective amount of a skin wellness ingredient.

15. The method of claim 14, further comprising applying the adhesive containing the skin wellness ingredient to at least one component selected from the group consisting of the top sheet material, the back sheet material, and the absorbent core.

16. The method of claim 14, further comprising preparing at least one elastic element and disposing the at least one elastic element between the top sheet material and the back sheet material.

17. The method of claim 16, further comprising adhesively bonding the at least one elastic element to at least one of the top sheet material, the back sheet material, and the absorbent core, wherein the adhesive comprises a skin care benefit effective amount of a skin wellness ingredient.

18. The method of claim 14, wherein the skin wellness ingredient is selected from the group consisting of algae extract, aloe, allantoin, bisabolol, calendula officinalis, Canadian balsam, cocoa butter, colloidal oatmeal, dimethicone, eucalyptus leaf oil, German chamomile, glycerin, Gorgonian extract, helichrysum angustifolium wax, jojoba, lanolin, lavandin, lemon balm, mineral oil, petrolatum, rosemary, spike lavender, tea tree, true lavender, Vitamin E, Vitamin E-acetate, witch hazel, or zinc oxide, and mixtures thereof.

19. The method of claim 14, wherein the amount of the skin wellness ingredient in the absorbent article is from about 1 weight % to about 3 wt %, based on the weight of the adhesive into which the skin wellness ingredient is incorporated.

20. The method of claim 14, wherein the amount of the skin wellness ingredient present in the absorbent article ranges from about 0.0003 weight % to about 0.15 weight %, based on the weight of the absorbent article.

21. The method of claim 14, comprising adhesively bonding the top sheet material to the back sheet material, whereby the adhesive comprises a skin care benefit effective amount of a skin wellness ingredient.

22. The method of claim 14, comprising adhesively bonding the absorbent core to either the top sheet material or the back sheet material, whereby the adhesive comprises a skin care benefit effective amount of a skin wellness ingredient.
